(19) United States
(12) Patent Application Publication
(10) Pub. No.: US 2026/0183474 A1
(43) Pub. Date: Jul. 2, 2026

(54) ANTI-BUBBLE DRIP CHAMBER

(71) Applicant: CareFusion 303, Inc., San Diego, CA (US)

(72) Inventors: Srinivas Ramakrishnarao PEDDI, Pune (IN); Ritesh Kantilal PARIKH, Pune (IN); Nandha KRISHNAN, Coimbatore (IN)

(21) Appl. No.: 19/006,641

(22) Filed: Dec. 31, 2024

(51) Int. Cl. A61M 5/14

(52) U.S. Cl. CPC A61M 5/1411

(57) ABSTRACT

Drip chambers are disclosed that can resist the formation of bubbles and turbulence in fluid moving through the drip chamber, and can include an inlet opening to an inner chamber formed at least in part by a sidewall having a portion that intersects a path of fluid droplets moving or falling from the inlet orifice into the inner chamber, where the path of fluid droplets causes the droplets to engage against a tangent impact plane at a location of the sidewall that is transverse relative to the path of fluid droplets such that the fluid droplets can slide from the impact plane into a pool of fluid within the inner chamber and exit the inner chamber through an outlet opening.

### BACKGROUND

The present disclosure generally relates to drip chambers used in the administration of fluids to patients through intravascular (“IV”) sets, and in particular, to drip chambers that resist the formation of bubbles in a fluid moving through the drip chamber.

Fluids are commonly administered into a patient's blood flow is through an IV set. An IV set is an apparatus that generally includes a connector for connection to a fluid reservoir, a drip chamber used to determine the flow rate of fluid from the fluid reservoir, tubing for providing a connection between the fluid reservoir and the patient, and a connector for attachment to a catheter that may be positioned intravenously in a patient. An IV set may also include a Y-connector that allows for the piggybacking of IV sets and for the administration of medicine from a syringe into the tubing of the IV set.

In conventional methods of administering a fluid into the blood vessel of a patient using an IV set, air is removed from IV set to resist or prevent complications for the patient. While this concern is critical when accessing arterial blood, it is also a concern when accessing the venous side. Specifically, if air, which can be in the form of bubbles, is permitted to enter a patient's blood stream while receiving the intravenous administration of liquids, the air bubbles can form an air embolism and cause serious injury to a patient, including organ damage, low blood pressure, and death.

In a majority of adults, the right atrium and the left atrium are completely separated from each other so that the blood and air bubbles are moved from the right atrium, to the right ventricle, and then to the lungs where the air bubbles may be safely vented. The bubble free blood is then returned to the left atrium, where the blood is moved to the left ventricle and then sent throughout the body.

However, in infants and some portion of the adult population, the right atrium and left atrium are not completely separated. Consequently, air bubbles can move directly from the right atrium into the left atrium and then be dispersed throughout the body. As a result, these air bubbles may cause strokes, tissue damage, and/or death. Therefore, it is important to prevent air bubbles from entering a patient's blood stream.

Although it is important to remove air bubbles while priming an IV set for use in the intravenous administration of liquids, the complete removal of air bubbles can be a time-consuming process. The process may also lead to contamination of the IV set by inadvertently touching a sterile portion of the IV set. Typically, when an IV set is primed, a clamp may be closed to prevent liquid from moving from a drip chamber through the tubing. The IV set can then be attached to an IV bag or bottle. Once attached, the drip chamber, which is typically made of a clear flexible plastic, may be squeezed to draw the liquid out of the IV bag or bottle and into the drip chamber. The drip chamber is allowed to fill to a level, e.g., one-half or one-third full, when the clamp is opened to allow liquid to flow through the tube to an end of the IV set.

This initial process, however, typically traps air in tubing which must be removed. For example, the flow of the liquid through the tubing of the IV set may be turbulent and can entrap air within the tube as the boundary layer between the liquid and the tubing is sheared. The flow rate out of the drip chamber may be higher than the flow rate of liquid entering the drip chamber. This can cause a bubble ladder to form as air is sucked from the drip chamber into the tubing.

Additionally, air bubbles may be formed as drops of liquid strike the surface of the pool of liquid within the drip chamber. These air bubbles can be pulled into the tubing of the IV set from the drip chamber. This problem may be aggravated in pediatric applications where the drip orifice may be smaller which may result in increased turbulence.

To remove air bubbles from the IV set, liquid from the IV bag or bottle may be allowed to flow through the tubing to encourage the air bubbles out the end of the IV set. As the liquid is allowed to flow out of the IV set to clear air bubbles from the tubing, the liquid may be directed to flow into a waste basket or other receptacle. During this procedure the end of the tubing may contact the waste basket or be touched by the attendant and thus, become contaminated. Another method to remove bubbles from the IV set is to directly remove air bubbles at a port in the IV set, such as a Y-connector, where the air bubbles may be removed using a syringe coupled to the port.

In some cases, a small pore filter may be used in the drip chamber to prevent air from entering the IV tubing from the drip chamber. However, the bubbles formed from the dripping action may become trapped on the filter, thus, reducing the flow of liquid through the filter to the IV tubing.

In some cases, a gap is minimized or eliminated between the surface of the pool of liquid within the drip chamber and an inlet opening of the drip chamber. However, minimizing or eliminating the gap between the surface of the liquid within the drip chamber and the inlet opening defeats the ability to monitor the flow rate and droplet size in the drip chamber.

### SUMMARY

The disclosed subject matter relates to drip chambers that can resist the formation of bubbles in a fluid moving through the drip chamber and can provide consistent or laminar fluid flow through the drip chamber.

In some embodiments, a drip chamber is disclosed comprising a top end portion, a bottom end portion opposite to the top end portion, and a sidewall extending between the top end portion and the bottom end portion, an inner chamber formed by the top end portion, the bottom end portion, and the sidewall, an inlet opening at the top end portion of the inner chamber, and an outlet opening at the bottom end portion of the inner chamber, wherein at least a portion of an inner surface of the sidewall intersects a path of a fluid dropping from the inlet opening into the chamber such that the portion of the inner surface forms an impact plane that is tangent to the path of the fluid dropping from the inlet opening into the chamber.

In some instances, methods for providing a drip chamber are disclosed as providing an inner chamber formed by a top end portion, a bottom end portion opposite to the top end portion, and a sidewall extending between the top end portion and the bottom end portion, wherein the top end portion comprises an inlet opening, and providing at least a portion of an inner surface of the sidewall that intersects a path of a fluid dropping from the inlet opening into the chamber to form an impact plane that is tangent to the path of the fluid dropping from the inlet opening into the chamber.

In certain embodiments, a drip chamber is disclosed and comprises a sidewall having an inner surface forming an inner chamber, an inlet opening at a top end portion of the inner chamber, and an outlet opening at a bottom end portion of the inner chamber, wherein the inlet opening forms an inlet opening axis that intersects a portion of the inner surface of the sidewall that extends radially inward.

Accordingly, the present application addresses several operational challenges in the administration of IV fluids to a patient, including the resisting the propensity for bubble formation in a drip chamber and providing consistent or laminar fluid flow through the drip chamber, while also increasing manufacturing complexity and reducing manufacturing costs of a drip chamber.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an embodiment of a drip chamber in use with an IV set coupled to a patient, in accordance with aspects of the present disclosure.

FIG. 2 is a front perspective view of a drip chamber, in accordance with aspects of the present disclosure.

FIG. 3 is a front cross-sectional view of the drip chamber of FIG. 2 along the line 3-3, in accordance with various aspects of the present disclosure.

FIG. 4 is an enlarged detail view of FIG. 3, in accordance with various aspects of the present disclosure.

FIG. 5 is a front cross-sectional view of the drip chamber of FIG. 3 coupled to an IV bag and IV tubing, in accordance with various aspects of the present disclosure.

FIG. 6 is a front cross-sectional view of another embodiment of a drip chamber in accordance with aspects of the present disclosure.

FIG. 7 is a front cross-sectional view of another embodiment of a drip chamber in accordance with aspects of the present disclosure.

FIG. 8 is a front cross-sectional view of another embodiment of a drip chamber in accordance with aspects of the present disclosure.

### DETAILED DESCRIPTION

The disclosed intravenous monitoring systems incorporate various features to facilitate the detection of characteristics and evaluation of several aspects of venous access, including proper position of a catheter inside a vein, fluid pathway occlusion or patency, and catheter extravasation or infiltration.

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology. Further, while the present description sets forth specific details of various embodiments, it will be appreciated that the description is illustrative only and should not be construed in any way as limiting.

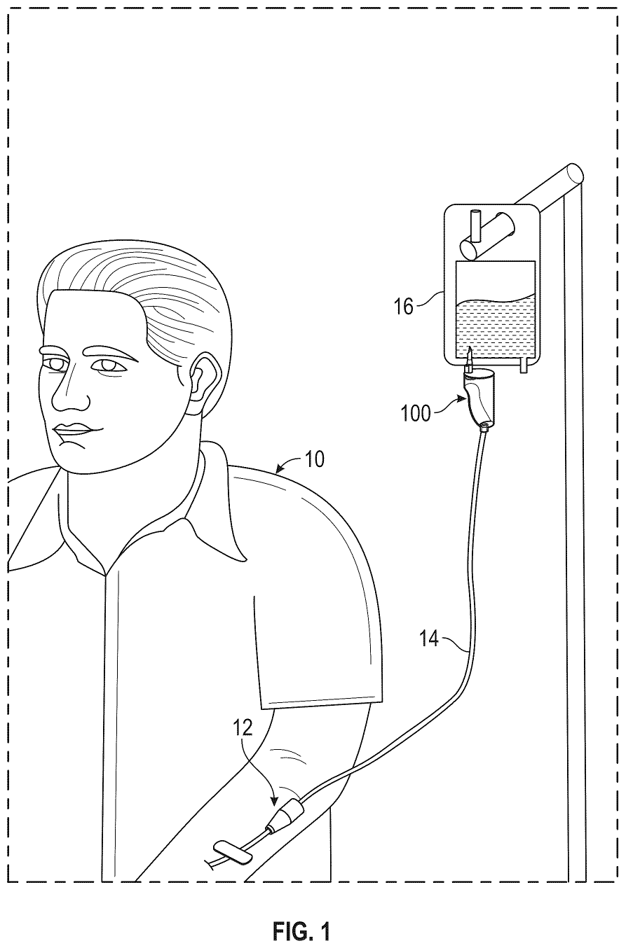

Referring now to the drawings, FIG. 1 illustrates an example of a drip chamber 100 coupled with a patient 10, according to some embodiments. The drip chamber 100 is part of an IV set that includes a catheter 12 and tubing 14 that fluidically couples the drip chamber 100 to the catheter 12. The drip chamber 100 is coupled to a IV bag 16 such that a fluid in the IV bag 16 can drip into the drip chamber 100, and move through the tubing 14 toward the patient.

The drip chamber 100 of the present disclosure is configured so that the droplets of fluid from an IV bag can move through an inlet opening and fall into an inner chamber and impact a sidewall of the drip chamber 100 before contacting a pool of the fluid within the inner chamber. A droplet of fluid falling from the inlet opening into the chamber forms a fluid droplet path. The impact of the droplet of fluid against the sidewall of the drip chamber 100 occurs at a location in which a portion the sidewall of the drip chamber extends radially inward such that the fluid droplet path intersects the portion the sidewall. Thus, the droplet of fluid falling from the inlet opening into the inner chamber can strike a surface of the sidewall that forms a tangent plane that is transverse to the fluid droplet path. The droplet can then slide along the inner surface of the drip chamber, from the impact location toward an outlet opening.

The features of the present disclosure provide a smooth transfer of the droplet into the drip chamber such that the formation of air bubbles in minimized or eliminated, disturbance to the pool of fluid is minimized or eliminated, and the fluid flow through the drip chamber becomes laminar.

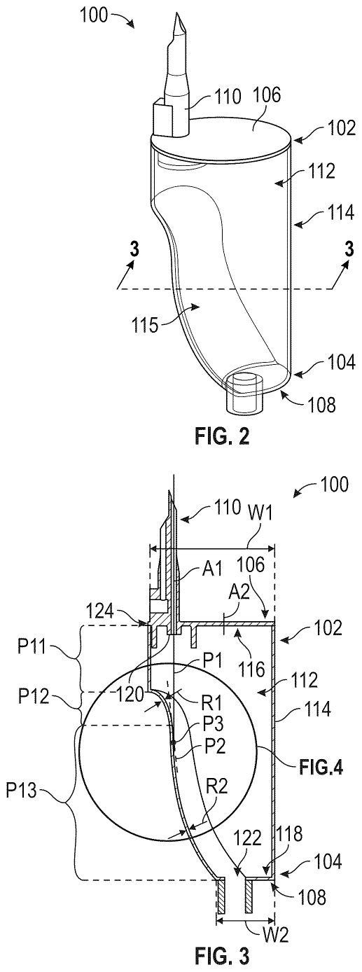

FIG. 2 is a front perspective view of a drip chamber 100, and FIG. 3 is a front cross-sectional view of the drip chamber of FIG. 2 along the line 3-3, according to the present disclosure. The drip chamber 100 includes a top end portion 102 and a bottom end portion 104, where the bottom end portion 104 is opposite to the top end portion 102. The top end portion 102 includes an outer top surface 106 of the drip chamber and an inlet opening forming a fluid passage into the drip chamber. The bottom end portion 104 includes an outer bottom surface 108 of the drip chamber and an outlet opening forming a fluid passage out of the drip chamber.

In some embodiments of the present disclosure, such as the embodiment shown in FIG. 2, the top end portion 102 of the drip chamber can include an IV bag spike 110. The IV bag spike 110 extends in a direction away from the outer top surface 106 and includes a fluid passage that is fluidically coupled with an inner chamber 112 of the drip chamber. The IV bag spike 110 is configured to be inserted into an IV bag 16 to form an inlet fluid pathway from the IV bag 16, through the IV bag spike 110, to an inner chamber 112 of the drip chamber.

An inner chamber 112 of the drip chamber is formed by inner surfaces of the top end portion 102, the bottom end portion 104, and a sidewall 114 of the drip chamber. The sidewall 114 of the drip chamber extends from an inner top surface 116 to an inner bottom surface 118 to form the inner chamber 112, which is configured to receive and retain a fluid therein.

To receive a fluid into the inner chamber 112, the drip chamber includes an inlet opening 120 to direct a fluid into the inner chamber 112. The inlet opening forms a portion of the inlet fluid pathway from the IV bag 16 into the inner chamber of the drip chamber. The drip chamber also includes an outlet opening 122 to direct a fluid out of the inner chamber 112. The outlet opening forms a portion of an outlet fluid pathway from the inner chamber 112 to tubing 14 of an IV set, where the tubing 14 can guide the fluid toward a patient.

The drip chamber 100 can receive a fluid from the inlet opening 120 into the inner chamber 112, and the outlet opening 122 can permit the fluid to exit the inner chamber 112. The diameter or cross-sectional area of the inlet opening 120 and the outlet opening 122 are configured so that a rate of fluid entering and exiting the inner chamber results in a volume of the fluid is retained to form a pool of fluid having a fluid fill level. In some embodiments of the present disclosure, the inlet opening 120 comprises a cross-sectional profile forming a first area, and the outlet opening 122 comprises a cross-sectional profile forming a second area, where the second area is less than the first area.

Additionally, the fluid forms droplets as the fluid moves through the inlet opening 120 into the inner chamber. The droplets of fluid fall from the inlet opening 120 into the inner chamber 112, forming a fluid droplet path P1. In some embodiments, the fluid droplet path P1 is coaxial with an inlet opening axis A1; however, the present disclosure also contemplates embodiments in which the inlet opening axis A1 may be spaced apart and/or transverse to the fluid droplet path P1.

As a fluid droplet falls from the inlet opening 120 into the inner chamber 112, the propensity to form bubbles in the volume of fluid within the inner chamber is resisted by the fluid droplet engaging against or striking a portion 115 of the inner surface of the sidewall that intersects the fluid droplet path P1. The location of the portion 115 of the inner surface of the sidewall that intersects the fluid droplet path P1 forms a tangent impact plane P2 that is transverse to the path of the fluid dropping from the inlet opening 120 into the chamber.

In some embodiments of the present disclosure, the impact plane P2 forms an acute angle or an obtuse angle, relative to any of the fluid droplet path P1 and the inlet opening axis A1. In some embodiments, the impact plane P2 is transverse, relative to any of the fluid droplet path P1 and the inlet opening axis A1.

To intersect the fluid droplet path P1, the portion 115 of the inner surface of the sidewall extends in a direction that is radially inward into or toward the fluid droplet path P1. The portion 115 of the inner surface of the sidewall can be distinct from another portion of the sidewall. For example, the drip chamber 100 can have a sidewall 114 forming a straight cylindrical wall, and the portion 115 of the inner surface of the sidewall can extend radially inward from the straight cylindrical wall toward the fluid droplet path P1.

The sidewall 114 can also be formed with more than one portion along a longitudinal length of the drip chamber defined between the outer top surface 106 and the outer bottom surface 108. The sidewall 114 can have a first portion P11, a second portion P12, and a third portion P13, where the first portion P11 extends from the outer top surface 106 toward the outer bottom surface 108, the second portion P12 extends from the first portion P11 toward the outer bottom surface 108, and the third portion P13 extends from the second portion P12 to the outer bottom surface 108.

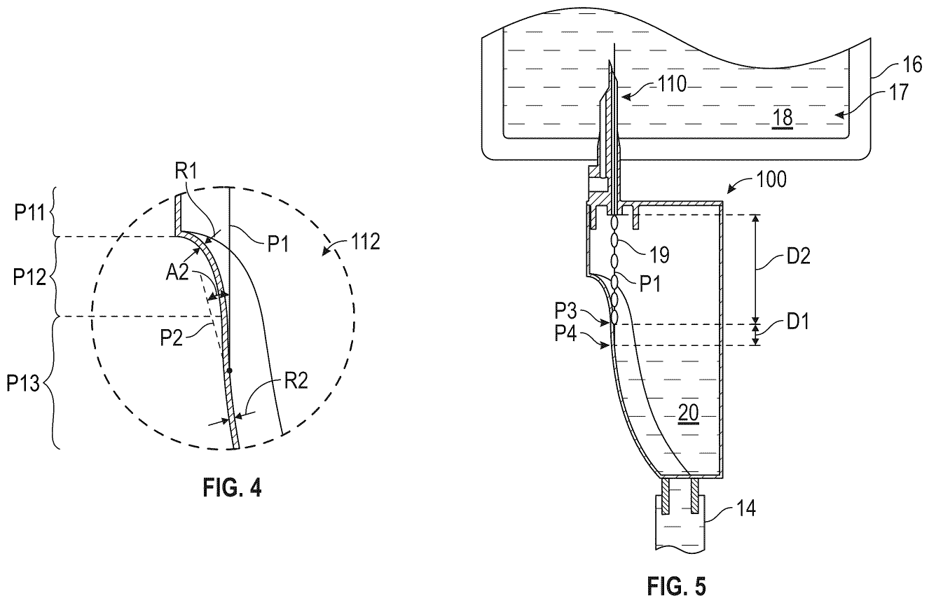

One or more of the first portion P11, the second portion P12, and the third portion P13 can form any of a convex and/or a concave surface. Referring to the example embodiment shown in FIGS. 2 and 3, and the detail view of FIG. 4, the first portion P11 forms a straight cylindrical wall that extends around the entire circumference of the drip chamber, the second portion P12 forms a convex inner surface of the sidewall, and the third portion P13 forms a concave inner surface of the sidewall. The third portion P13 of the sidewall 114 intersects the fluid droplet path P1, and the impact plane P2 forms an angle A2 that is greater than zero degrees and less than ninety degrees with the fluid droplet path P1. In some embodiments of the present disclosure, the impact plane P2 is formed along the first portion P11 and/or the second portion P12 of the sidewall.

The present disclosure also contemplates that each of the concave and convex surfaces of the drip chamber may be formed with a radius that varies along a length of each respective portion, in some embodiments of the present disclosure, the second portion P12 forms a first radius R1, and the third portion P13 forms a second radius R2. Referring to FIGS. 3 and 4, the second portion P12 forms a convex inner surface having a first radius R1, the third portion P13 forms a concave inner surface having a second radius R2, where the first radius R1 is smaller than the second radius R2.

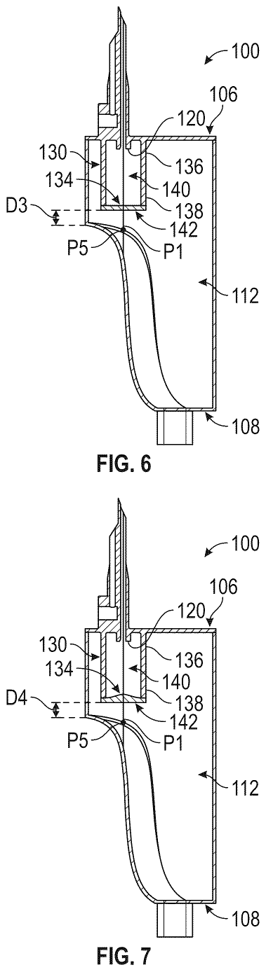

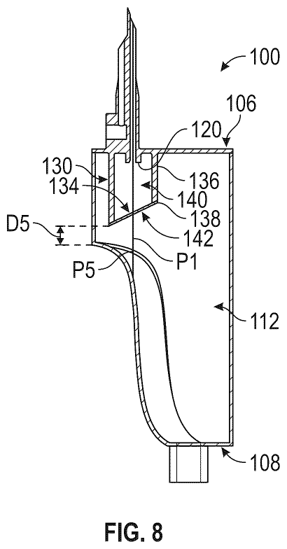

In some embodiments of the present disclosure, the second portion P12 of the sidewall 114, forming a convex inner surface of the sidewall (FIGS. 6, 7, and 8), intersects the fluid droplet path P1, and the impact plane P5 is tangent to the fluid droplet path P1.

The portion 115 of the inner surface of the sidewall extends partially around the circumference of the drip chamber, where the circumference is defined by a cross-sectional profile that is transverse to the longitudinal length of the drip chamber 100, thereby forming the drip chamber 100 with a cross-sectional profile that is asymmetrical. In some embodiments of the present disclosure, the portion 115 of the inner surface of the sidewall extends entirely around the circumference of the drip chamber, thereby forming a cross-sectional profile that is symmetrical.

To form the portion 115 of the inner surface of the sidewall that intersects the fluid droplet path P1, the inner chamber 112 can have a width, defined between opposite inner surfaces of the sidewall 114, that decreases in a direction from the top end portion 102 toward the bottom end portion 104. As such, the inner chamber 112 can have a first width W1 proximate to the top end portion 102 and a second width W2 proximate to the bottom end portion 104, where the first width W1 is greater than the second width W2.

To provide an impact plane P2 where a portion 115 of the inner surface of the sidewall that intersects the fluid droplet path P1, the inlet opening 120 of the drip chamber can be positioned proximate to the outer perimeter 124 of the inner top surface 116. In some embodiments of the present disclosure, a distance from the inlet opening 120 to the outer perimeter 124 is less than a distance from the inlet opening 120 to a central axis A2 of the inner top surface 116. In some embodiments of the present disclosure, the inlet opening 120 is positioned radially outward from the central axis A2.

The drip chamber 100 can be coupled with a fluid source, such as an IV bag 16, and tubing, such as the tubing 14 of an IV set, as shown in FIG. 5. To couple the drip chamber 100 with an IV bag 16, the IV bag spike 110 can be inserted into the IV bag 16 to form a fluid pathway from a fluid chamber 17 in the IV bag, through the inlet opening 120, to the inner chamber 112 of the drip chamber. Fluid 18 from the IV bag can move through the IV bag spike 110 and inlet opening 120 of the drip chamber. As the fluid 18 moves through the inlet opening 120, the fluid 18 forms droplets 19 that fall into the inner chamber 112. The path of the falling droplets 19 forms a fluid droplet path P1. Because the drip chamber 100 is configured with at least a portion 115 of the sidewall extending into the fluid droplet path P1, the droplets 19 strike against or contact the portion 115 of the sidewall before striking against the surface of the pool 20 of the fluid within the inner chamber.

The impact plane P2 is a tangent plane located an impact level P3, where impact level P3 is between the inner top surface 116 of the drip chamber and a fluid fill level P4 of the drip chamber. A distance D1 between impact level P3 and the fluid fill level P4 is greater than zero. Because the impact plane P2, where the portion 115 of the inner surface of the sidewall intersects the fluid droplet path P1, is transverse to the fluid droplet path P1, the droplets 19 strike against or contact the impact plane P2 and slide along the sidewall toward the outlet opening 122. As a droplet 19 slides along the sidewall, the velocity of the droplet 19 is reduced due, at least in part, to friction between the droplet 19 and the inner surface of the sidewall. The droplet 19 can then slide along the sidewall until the droplet contacts or enters the pool 20 of the fluid within the inner chamber.

The impact level P3 is also spaced apart from the inlet opening 120 by a distance D2 that is greater than zero. In some embodiments, the distance between the inlet opening 120 and the impact level P3 is greater that a height or diameter of droplet 19. In some examples, the distance between the inlet opening 120 and the impact level P3 is at least two times the height or diameter of droplet 19. The distance between the inlet opening 120 and the impact level P3 permits observation of the flow rate and size of the droplets 19 falling from the inlet opening 120 to the impact plane P2. Thus, the features of the present disclosure can provide a drip chamber 100 that can reduce or eliminate turbulent characteristics of fluid entering the inner chamber 112 and can provide for the smooth transfer of a fluid droplet 19 into the pool of fluid within the inner chamber 112, which can minimize or eliminate disturbance in the fluid and reduce the propensity for bubble formation in the fluid.

Referring to FIGS. 6, 7, and 8, the drip chamber 100 can include a filter 130 at the inlet opening 120 so that the filter can receive the fluid moving through the inlet opening 120 toward the inner chamber 112. Filtration of the fluid upon entering the drip chamber and before contacting the impact plane P2 can provide for consistent fluid flow characteristic through the drip chamber 100 and to the patient.

The filter 130 can include a wall and a droplet surface 134. The wall has a proximal end 136 attached to the top end portion 102 of the drip chamber and a distal end 138 forming the droplet surface 134. A filter cavity 140 is formed by the inner surface of the wall and extends from the droplet surface 134 through the proximal end 136 of the filter.

The droplet surface 134 is formed by an inner surface of a filter material that faces toward the filter cavity 140. The filter material can be positioned at the distal end 138 of the filter, or at another location between the proximal and distal ends of the filter such that a fluid engages against the filter material as the fluid moves through the inlet opening 120 toward the inner chamber 112.

As a fluid moves through the inlet opening 120 toward the inner chamber 112, the fluid can engage against the droplet surface 134 of the filter. The droplet surface 134 can formed as a planar surface, as shown in FIG. 6. In another embodiment of the present disclosure, the droplet surface 134, 135 can formed as convex surface, as shown in FIG. 7. In any embodiment of the present disclosure, the filter material can be positioned so that the droplet surface 134, 135 is oriented transverse, relative to the fluid droplet path P1.

The fluid exits the filter 130 and drops from the filter toward the impact plane P2, P5. In some embodiments of the present disclosure, the fluid exits the filter at an outer surface 142 of the filter material. In any embodiment of the present disclosure, the point at which the fluid exits the filter, such as the outer surface 142 of the filter material, is spaced apart from the impact plane P2, P5 by a distance D3, D4, D5 that is greater than zero. In some examples, the distance D3, D4, D5 is at least two times the height or diameter of droplet 19. The distance D3, D4, D5 between the point at which the fluid exits the filter and the impact plane P2, P5 permits observation of the flow rate and size of the droplets 19 falling from the filter 130 to the impact plane P2.

Various examples of aspects of the subject technology are described below for convenience. These are provided as examples and do not limit the subject technology. Further, the examples may be combined in any combination, and placed into respective embodiments.

A drip chamber comprising a top end portion, a bottom end portion opposite to the top end portion, and a sidewall extending between the top end portion and the bottom end portion, an inner chamber formed by the top end portion, the bottom end portion, and the sidewall, an inlet opening at the top end portion of the inner chamber, and an outlet opening at the bottom end portion of the inner chamber, wherein at least a portion of an inner surface of the sidewall intersects a path of a fluid dropping from the inlet opening into the chamber such that the portion of the inner surface forms an impact plane that is tangent to the path of the fluid dropping from the inlet opening into the chamber.

Wherein the impact plane is transverse, relative to the path of a fluid dropping from the inlet opening into the chamber.

Wherein the top end portion comprises a outer surface having a central axis, and wherein the inlet opening is positioned radially outward from the central axis.

A drip chamber wherein comprising a filter fluidically coupled to the inlet opening. Wherein the filter is concentric with the inlet opening. Wherein the filter comprises a droplet surface that is axially aligned with the inlet opening. Wherein the droplet surface forms a convex surface. Wherein the droplet surface is transverse, relative to a path of a fluid dropping from the inlet opening into the chamber.

Wherein a width of the inner chamber, defined between opposing inner surfaces of the inner chamber, decreases in a direction from the top end portion toward the bottom end portion.

A drip chamber comprising a bag spike extending from an outer surface of the top end portion in a direction opposite to the bottom end portion.

Wherein the sidewall comprises a first portion, a second portion, and a third portion, and wherein the first portion extends from the top end portion toward the bottom end portion, the second portion extends from the first portion toward the bottom end portion, and the third portion extends from the second portion to the bottom end portion.

Wherein the impact plane is formed along the third portion of the sidewall.

Wherein the inner surface of the sidewall along the second portion forms any of a convex surface or a concave surface.

Wherein the inner surface of the sidewall along the third portion forms the other of the convex surface or the concave surface.

Wherein the convex surface comprises a first segment, a second segment, and an apex, the first segment extending in a first direction from the first portion of the sidewall into the inner chamber to the apex, and the second segment extending in a second direction from the apex toward the third portion of the sidewall.

Wherein the concave surface comprises a first segment, a second segment, and an apex, the first segment extending in a first direction from the first portion of the sidewall toward the third portion of the sidewall to the apex, and the second segment extending in a second direction from the apex into the inner chamber.

Wherein a cross-sectional profile of the sidewall is asymmetrical.

A method of providing a drip chamber comprising providing an inner chamber formed by a top end portion, a bottom end portion opposite to the top end portion, and a sidewall extending between the top end portion and the bottom end portion, wherein the top end portion comprises an inlet opening, and providing at least a portion of an inner surface of the sidewall that intersects a path of a fluid dropping from the inlet opening into the chamber to form an impact plane that is tangent to the path of the fluid dropping from the inlet opening into the chamber.

Wherein the drip chamber comprises a filter fluidically coupled to the inlet opening.

Wherein providing the filter comprises the filter having a droplet surface that is axially aligned with the inlet opening.

The drip chamber further comprising providing any of a concave surface or a convex surface along a portion of the inner surface of the sidewall forming the impact plane.

Wherein impact plane is between the inlet opening and a fluid fill level of the inner chamber.

Wherein the sidewall comprise a cross-sectional profile that is asymmetrical.

A drip chamber comprising a sidewall having an inner surface forming an inner chamber, an inlet opening at a top end portion of the inner chamber, and an outlet opening at a bottom end portion of the inner chamber, wherein the inlet opening forms an inlet opening axis that intersects a portion of the inner surface of the sidewall that extends radially inward.

Wherein the portion of the inner surface of the sidewall, at the intersection with the inlet opening axis, forms an impact plane that is at an acute angle or an obtuse angle, relative to the inlet opening axis.

Wherein the portion of the inner surface of the sidewall forms any of a convex surface or a concave surface.

The drip chamber comprising a filter fluidically coupled to the inlet opening. Wherein the filter comprises a droplet surface that is axially aligned with the inlet opening. Wherein the droplet surface is transverse, relative to the inlet opening axis.

Wherein a width of the inner chamber, defined between opposing inner surfaces of the inner chamber, decreases in a direction from the inlet opening toward the outlet opening.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A drip chamber comprising:
a top end portion, a bottom end portion opposite to the top end portion, and a sidewall extending between the top end portion and the bottom end portion;
an inner chamber formed by the top end portion, the bottom end portion, and the sidewall;
an inlet opening at the top end portion of the inner chamber; and
an outlet opening at the bottom end portion of the inner chamber;
wherein at least a portion of an inner surface of the sidewall intersects a path of a fluid dropping from the inlet opening into the inner chamber such that the portion of the inner surface forms an impact plane that is tangent to the path of the fluid dropping from the inlet opening into the inner chamber.

2. The drip chamber of claim 1, wherein the impact plane is transverse, relative to the path of a fluid dropping from the inlet opening into the chamber.

3. The drip chamber of claim 1, wherein the top end portion comprises a outer surface having a central axis, and wherein the inlet opening is positioned radially outward from the central axis.

4. The drip chamber of claim 1, further comprising a filter fluidically coupled to the inlet opening.

5. The drip chamber of claim 4, wherein the filter comprises a droplet surface having a convex surface.

6. The drip chamber of claim 4, wherein the filter comprises a droplet surface that is transverse, relative to a path of a fluid dropping from the inlet opening into the chamber.

7. The drip chamber of claim 1, wherein a width of the inner chamber, defined between opposing inner surfaces of the inner chamber, decreases in a direction from the top end portion toward the bottom end portion.

8. The drip chamber of claim 1, further comprising a bag spike extending from an outer surface of the top end portion in a direction opposite to the bottom end portion.

9. The drip chamber of claim 1, wherein the sidewall comprises a first portion, a second portion, and a third portion, and wherein the first portion extends from the top end portion toward the bottom end portion, the second portion extends from the first portion toward the bottom end portion, and the third portion extends from the second portion to the bottom end portion.

10. The drip chamber of claim 9, wherein the impact plane is formed along the third portion of the sidewall.

11. The drip chamber of claim 9, wherein the inner surface of the sidewall along the second portion forms any of a convex surface or a concave surface.

12. The drip chamber of claim 11, wherein the inner surface of the sidewall along the third portion forms the other of the convex surface or the concave surface.

13. A method of providing a drip chamber comprising:
providing an inner chamber formed by a top end portion, a bottom end portion opposite to the top end portion, and a sidewall extending between the top end portion and the bottom end portion, wherein the top end portion comprises an inlet opening; and
providing at least a portion of an inner surface of the sidewall that intersects a path of a fluid dropping from the inlet opening into the inner chamber to form an impact plane that is tangent to the path of the fluid dropping from the inlet opening into the inner chamber.

14. The method of claim 13, further comprising providing a filter coupled to the inlet opening and comprising a droplet surface that is axially aligned with the inlet opening.

15. The method of claim 13, further comprising providing any of a concave surface or a convex surface along a portion of the inner surface of the sidewall forming the impact plane.

16. The method of claim 13, wherein the impact plane is between the inlet opening and a fluid fill level of the inner chamber.

17. A drip chamber comprising:
a sidewall having an inner surface forming an inner chamber;
an inlet opening at a top end portion of the inner chamber; and
an outlet opening at a bottom end portion of the inner chamber;
wherein the inlet opening forms an inlet opening axis that intersects a portion of the inner surface of the sidewall that extends radially inward.

18. The drip chamber of claim 17, wherein the portion of the inner surface of the sidewall, at the intersection with the inlet opening axis, forms an impact plane that is any of an acute angle or an obtuse angle, relative to the inlet opening axis.

19. The drip chamber of claim 17, further comprising a filter coupled to the inlet opening.

20. The drip chamber of claim 19, wherein the filter comprises a droplet surface that is axially aligned with the inlet opening.
